(19) United States
(12) Patent Application Publication
(10) Pub. No.: US 2015/0314012 A1
(43) Pub. Date: Nov. 5, 2015

(54) Protein Conjugates

(71) Applicant: BIO-KER S.R.L, Milano (IT)

(72) Inventors: Giancarlo Tonon, Pula (IT); Gaetano Orsini, Gallarate (IT); Rodolfo Schrepfer, Villa Guardia (IT)

(21) Appl. No.: 14/650,962

(22) PCT Filed: Dec. 10, 2013
(86) PCT No.: PCT/EP2013/076060

§ 371 Date: Jun. 10, 2015

(30) Foreign Application Priority Data
Dec. 10, 2012 (IT) MI2012A002097

(51) Int. Cl. A61K 47/48; C12N 9/96; C12N 9/10; A61K 38/19

(52) U.S. Cl. CPC A61K 47/48269; A61K 38/193; C12N 9/96; C12N 9/1044; C12Y 203/02013

(57) ABSTRACT

The present invention concerns the field of conjugated peptides suitable for the production of drugs having an improved plasma half-life. In particular the present invention relates to a conjugated protein, obtained by an enzymatic reaction via microbial transglutaminase (MTGase), and an improved process for removing residual transglutaminase from peptides or recombinant proteins enzymatically conjugated by microbial transglutaminase (MTGase) to hydrophilic non-immunogenic polymer at a glutamine side-chain through an amidic linkage, allowing to obtain purified conjugated peptides or proteins which are stable against the enzymatic hydrolysis of the amidic bond between the peptide or protein moiety and the hydrophilic polymer and being free from product derived degradation displays the stability required for a drug.

### FIELD OF THE INVENTION

The present invention concerns the field of conjugated peptides suitable for the production of drugs having an improved plasma half-life. In particular the present invention relates to a conjugated protein, obtained by an enzymatic reaction via microbial transglutaminase (MTGase), and an improved process for the preparation of highly pure conjugated proteins, which are free from product derived degradation and exhibit an improved shelf-life.

### STATE OF THE ART

The conjugation to biocompatible, high molecular weight polymers is one of the most applied technologies for increasing the half-life of therapeutic peptides or proteins and for improving their long lasting effect. In particular, in the so-called PEGylation reaction, which has been extensively employed, the chosen protein is covalently bound to one or more linear or branched poly-(ethylene glycol) (PEG) chains with a molecular weight ranging from 1,000-2,000 Daltons (Da) to 20,000-40,000 Da or even higher. In general, PEGylated proteins show lower renal clearance rates, as well as higher stability and reduced immunogenicity. When a polypeptide is suitably bound to PEG its hydrodynamic volume increases and its physico-chemical properties are modified, while fundamental biological functions, such as in vitro activity or receptor recognition, may remain unchanged, undergo a reduction or, in some cases, be completely eradicated. PEG conjugation masks the protein surface and increases its apparent molecular size, thus decreasing renal ultrafiltration, preventing interactions with antibody or antigen processing cells and reducing proteolytic degradation. Finally, PEG conjugation confers to the PEGylated molecules its physico-chemical properties and therefore peptide and non-peptide drug biodistribution and solubility are modified, too. Another option for protein conjugation, as an alternative to PEG, is employing other linear or branched biocompatible polymers, such as, for instance, dextran, poly(vinylpyrrolidone), poly(acryloylmorpholine), polysaccharides, and so on. PEGylation is commonly performed by chemical reactions between aminoacid reactive side-chains and a suitably functionalized methoxy-PEG (m-PEG). Commonly employed chemical PEGylation techniques are described in the following publications:
S. Zalipsky, Chemistry of Polyethylene Glycol Conjugates with Biologically Active Molecules, Adv. Drug Deliv. Rev., 16, 157-182, 1995;
F. M. Veronese, Peptide and Protein PEGylation: a Review of Problems and Solutions, Biomaterials, 22, 405-417, 2001.
S. Jevs\check{e}var, M. Kunstelj, V. G. Porekar, PEGylation of therapeutic proteins, Biotechnol. J. 5, 113-128, 2010.

Other than chemical PEGylation, enzymatic procedures to bind m-PEG chains and proteins have been described. These are based for instance on the employment of transglutaminase enzymes, both of prokaryotic and eukaryotic origin, to catalyze transfer of m-PEG, functionalized with a primary amino group, to the acyl groups of glutamine residues, naturally present in the polypeptide chain of interest or inserted by site-specific mutagenesis reactions (H. Sato, Enzymatic Procedure for Site-Specific PEGylation of Proteins, Adv. Drug Deliv. Rev., 54, 487-504, 2002).

For instance, both EP785276 and U.S. Pat. No. 6,010,871 describe the use of a microbial transglutaminase (MTGase) to link polymer chains to peptides and proteins with at least one glutamine residue in their aminoacid sequence. In these patents, although examples are given of mono-substitution on some model proteins, it is not clear if the substitutions are site-specific too, which means whether they yield a single molecular form or a positional isomer mixture where, though mono-substituted, the polymer chain is bound to different glutamines.

On the other hand, both EP2049566 and U.S. Pat. No. 7,893,019 disclose a new G-CSF analogue selectively monopegylated at glutamine 135 by enzymatic reaction using MTGase.

However, no attention in the above reported patents and papers is paid to the purification process of the PEGylated product resulting from the enzymatic reaction, in particular considering the amount of the contaminating residual MTGase in the product which unexpectedly could still hydrolyze the conjugated PEG molecule and give place to product derived heterogeneity.

The need and importance is increasingly felt for the development of a process which allows to maintain stable therapeutic proteins.

It is therefore object of the present invention the development of a process which allows to reduce the residual MTGase contamination which is present after conjugation reactions and which degrades the conjugated drug during its storage.

### SUMMARY OF THE INVENTION

The present invention concerns a conjugated protein, obtained by an enzymatic reaction via microbial transglutaminase (MTGase), characterized by the fact that the content of residual MTGase in the purified product is not higher than 3 p.p.m, said conjugated protein exhibiting a shelf-life, at a temperature in the range from 2 to 8° C., of at least 36 months.

In a further aspect the invention concerns a process for the purification of a conjugated protein, obtained by enzymatic reaction via transglutaminase, by cation exchange chromatography.

As will be further described in the detailed description of the invention, the process of the present invention has the advantages of allowing to obtain highly pure conjugated therapeutic proteins.

The process for the purification of a conjugated protein according to the present invention includes the steps of:

a. bringing a cation exchange chromatography column to a pH of less than 4;
b. loading the chromatography column with a reaction mixture containing the PEGylated protein, having a pH of less than 4 on the column of step a.;
c. eluting the chromatography column of step b. with an eluent having a pH of less than 4,
thereby collecting a fraction containing the conjugated protein having a residual microbial transglutaminase content lower or equal to 3 ppm of the total amount of the conjugated protein, said conjugated protein exhibiting a shelf-life of at least 36 months, at a temperature in the range from 2 to 8° C.

A further aspect of the present invention is a conjugated protein obtainable by the process herein described and a pharmaceutical composition comprising said conjugated protein and pharmaceutically acceptable excipients.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-8, wherein:

FIG. 1: shows an RP-HPLC fluorimetric assay of MTGase. RP-HPLC fluorimetric assay of MTGase. Calibration curve (a) and RP-HPLC separation of fluorescent substrate CBZ-Gln-Gly-CAD-DNS at 7.3 min from fluorescent product CBZ-Gln(Gly-CAD-DNS)-Q-NH-CH2-CH2-O-Me at 8.0 min (b)

FIG. 2: shows SE-HPLC chromatograms of Met-G-CSF and mPEG-NH2 20 kDa reaction mixture in the presence of MTGase after about 30 min (a) and after 16 hour (b) showing that Met-G-CSF (eluted with a retention time of 11.5 min) is pegylated to give Met-G-CSF-Gln135-PEG 20 kDa (retention time 7.9 min). Peak eluted at 13 min is due to solvent front

FIG. 3: shows the elution profile of MTGase from a Macrocap SP column at pH 5

FIG. 4: shows the elution profile of PEGylated Met-G-CSF and MTGase from a Macrocap SP column at pH5.

FIG. 5: shows the RP-HPLC fluorimetric assay of residual MTGase in MTGase separation fractions 26-40 (a) and in PEGylated Met-GCSF+MTGase fractions 21-58 (b) and fractions 66-75 (c).

FIG. 6: shows the IE-HPLC of purified r-Met-G-CSF-Q135-PEG 20 kDa before (a) and after (b) storage for 1 month at 5° C. in the presence of 50 ppm of MTGase. The distorted peak of Met-G-CSF eluted at 7.5-8.1 min is an artifact due to the elution with the front of the solvent

FIG. 7: shows the IE-HPLC of purified r-Met-G-CSF before (a) and after (b) 1.5 hour treatment with 50 ppm of MTGase at room temperature.

FIG. 8: shows the stability data of Met-G-CSF-Gln135-PEG produced by TGase mediated pegylation and purified by cation exchange chromatography at different pH.

### DETAILED DESCRIPTION OF THE INVENTION

The present invention concerns a conjugated protein, obtained by an enzymatic reaction via microbial transglutaminase (MTGase), characterized by the fact that the content of residual MTGase in the purified product is not higher than 5 p.p.m, preferably 3 p.p.m, said conjugated protein exhibiting a shelf-life of at least 24 months, preferably 36 months, at a temperature in the range from 2 to 8° C.

A purification process for obtaining an highly pure therapeutic peptide or recombinant protein conjugated with a hydrophilic non-immunogenic polymer through amidic linkage to glutamine using microbial transglutaminase (M-TGase) catalysed reaction that allows to obtain a conjugated peptide or protein containing very low amount of residual contaminating enzyme (even as low as 3 ppm).

Advantageously the conjugated (preferably PEGylated) peptide or protein has been proved to show a very high stability, for not less than 36 months when stored at 5±3° C., therefore can be used as drug. Due to the unexpected high purity the cleavage of enzyme catalyzed amide bond linkage between peptide or protein and the polymer cannot occur, consequently providing a stable product which may be advantageously used as a drug.

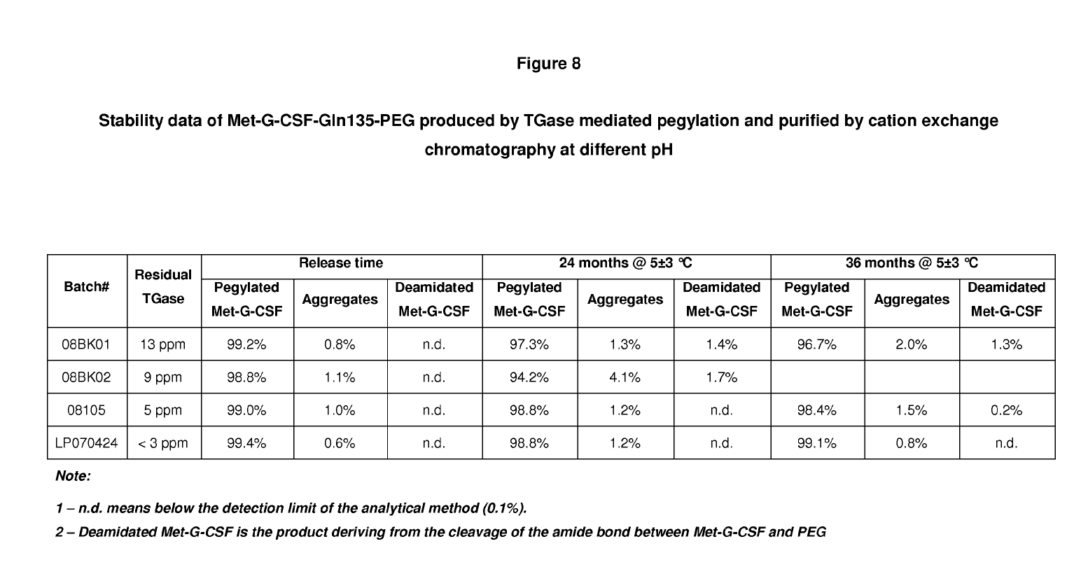

As an example demonstrating the advantage of the purification performed at pH lower than 4.0, in FIG. 8 there are reported the stability data on 4 batches of pegylated Met-G-CSF manufactured by TGase pegylation, purified by cation exchange chromatography at different pH and stored in refrigerator for 24 and 36 months. Batches 08BK01 and 08BK02, purified at pH higher than 4.0 and containing more than 3 ppm of residual TGase, show an high amount of deamidated Met-G-CSF (>1%) after 24 months of storage. Batch 08105, which was purified at pH 4.0 and containing 5 ppm of residual transglutaminase, although more stable than batches 08BK01 and 08BK02, shows a significant amount of deamidated Met-G-CSF (0.2%) after 36 months of storage. Batch LP070424, which was purified at pH lower than 4.0 and containing no detectable amounts of residual TGase (<3 ppm) is the most stable one and don't show detectable content of deamidated Met-G-CSF also after 36 months of storage in refrigerator.

In a preferred aspect the conjugated protein according to the present invention is obtained through an enzymatic reaction catalyzed by microbial transglutaminase, between a therapeutic protein and a hydrophilic polymer, preferably a hydrophilic non immunogenic polymer.

The therapeutic protein can be preferably selected from the group consisting of Met-G-CSF, G-CSF, GM-CSF, h-GH, Interferons, interleukins, Fab and scFv antibody fragments, Glucagon, GLP-1, Insulins and derivatives and analogues thereof.

The hydrophilic non immunogenic polymer can be, in a preferred form, selected from the group consisting of polyethyleneglycol, polyacryloyl morpholine, polyvinyl pyrrolidone, and hydroxyl ethyl starch.

In a more preferred aspect, the conjugated protein is obtained through the enzymatic reaction catalyzed by microbial transglutaminase, between Met-G-CSF and amino-polyethyleneglycol, thereby allowing to obtain Met-G-CSF-Gln135-PEG.

The conjugated proteins according to the present invention have the advantage of exhibiting a shelf-life, at a temperature in the range from 2 to 8° C., preferably of 5° C., of at least 36 months.

Such a shelf-life was unexpected and has been previously not been obtained by the proteins of the prior art, which have a residual MTGase contamination which is present after conjugation reactions and which degrades the conjugated drug during its storage (FIG. 8).

The present invention concerns a process for the purification of a conjugated protein, obtained by an enzymatic reaction via transglutaminase, by cation exchange chromatography including the steps of,

a. bringing a cation exchange chromatography column to a pH of less than 4;
b. loading the chromatography column with a reaction mixture containing the conjugated protein, having a pH of less than 4 on the column of step a.;
c. eluting the chromatography column of step b. with an eluent having a pH of less than 4,
thereby collecting a fraction containing the conjugated protein having a residual microbial transglutaminase content lower or equal to 3 ppm of the total amount of the conjugated protein, said conjugated protein exhibiting a shelf-life of at least 36 months, at a temperature in the range from 2 to 8° C.

The method of the present invention has the advantage of allowing to obtain a PEGylated protein with a high degree of purity and a residual microbial transglutaminase content lower or equal to 3 ppm, which in turn allows a significant improvement in drug stability. In the method provided by the present invention the transglutaminase is separated from the PEGylated product in a surprisingly efficient manner. This unexpected result is obtained by performing the cation exchange purification of the conjugated peptide or protein from reagents and contaminating enzyme, using an acidic eluent at pH<4.0. At this pH value there are no non-covalent interactions between the PEGylated protein and the enzyme, and therefore the retention of part of the enzyme by the PEGylated protein is avoided.

In a preferred aspect, the pH of steps a., b. and c. of the process according to the present invention is in the range of from 3 to 3.9, preferably pH 3.8, even more preferably pH 3.5.

For the purposes of the present invention:
The term “conjugated protein” refers to a protein or a peptide which is covalently attached to a hydrophilic polymer, preferably a hydrophilic non immunogenic polymer. For example the hydrophilic non immunogenic polymer can be polyethyleneglycol, polyacryloyl morpholine, polyvinyl pyrrolidone, and hydroxyl ethyl starch.
The term “Met-G-CSF-Gln135-PEG refers to methionylated granulocyte colony stimulating factor conjugated to polyethyleneglycol at glutamine 135.
The term “PEGylated protein or PEGylated peptide” refers to a protein or a peptide which is covalently attached to a polyethylene glycol (PEG) polymer chains.
The term “therapeutic peptide or protein derivative” refers to an aminoacid chain maintaining wholly or partially the biological activity of the native sequence.
The term “protein or peptide or their homologues” refers to protein or peptide variants with aminoacid sequence at least 90% identical to the aminoacid sequence of corresponding native peptides or proteins. In this context aminoacid sequence variations of protein or peptide can be due to addition, subtraction, substitution or chemical modification of one or more aminoacids of the native sequence.
The term “suitable biocompatible polymer” refers to any polymer employed for the enzymatic conjugation reaction and implies that the same conjugated polymer, when administered through the systemic route do not induce immune activation, nor significantly cause specific antipolymer antibodies. Example of biocompatible polymers included in the present invention, are polyethylene glycols (PEGs), polyoxypropylenes, polyvinylpyrrolidones, polyacryloylmorpholines, polysaccharides and dextrans.
The term “therapeutic peptide or recombinant protein conjugated to a biocompatible polymer by microbial transglutaminase (MTGase) catalysed reaction” refers to any clinically useful protein or peptide as well as to their homologues or variants which are covalently linked to a suitable biocompatible polymer by using MTGase in order to increase the peptide or protein half-life. The term therapeutic peptide refers to aminoacid sequences of less than 50 residues prepared by chemical synthesis or by recombinant DNA technology.

In a more preferred aspect, the pH of steps a. and b. of the process according to the present invention is obtained with an acetate buffer, preferably a 30 mM acetate buffer.

In a still more preferred aspect, the pH of step c. of the process according to the present invention is obtained with an acetate buffer, preferably a 200 mM acetate buffer.

In a further aspect, after the loading step b. of the process according to the present invention, the chromatography column is washed with an acetate buffer, preferably a 30 mM acetate buffer, in a volume which is of 4 times the volume of the chromatography column.

In a further aspect, the reaction mixture of step b. of the process according to the present invention, is obtained through an enzymatic reaction catalyzed by microbial transglutaminase, between a therapeutic protein and a hydrophilic polymer, preferably a hydrophilic non immunogenic polymer.

In a further aspect of the present invention, the therapeutic protein is selected from the group consisting of granulocyte colony-stimulating factor (G-CSF) and its clinically used variants such as Met-G-CSF (Filgrastim), granulocyte macrophage colony-stimulating factor (GM-CSF); interferons (IFNs); human growth hormone (h-GH); interleukins, monoclonal antibody fragments such as Fab and scFv fragments; insulins; glucagon and incretin mimetic peptides such as glucagon-like peptide 1 (GLP-1), exenatide and derivatives and analogues thereof.

In a further aspect of the present invention, the hydrophilic non immunogenic polymer is selected from the group consisting of polyethyleneglycol (PEG), polyacryloyl morpholine (PAM), polyvinyl pyrrolidone (PVP), and hydroxyl ethyl starch.

In a more preferred aspect, the conjugated protein obtained by the process according to the present invention is a PEGylated protein and is obtained through an enzymatic reaction catalyzed by microbial transglutaminase, between Met-G-CSF and amino-polyethyleneglycol, thereby allowing to obtain Met-G-CSF-Gln135-PEG.

The process according to the present invention advantageously allows to obtain a conjugated protein which exhibits a shelf-life, at a temperature of 5° C., of at least 36 months.

In a further aspect the present invention regards a conjugated protein, preferably a PEGylated protein obtained by the process described herein.

The conjugated protein which is obtainable by the cation exchange chromatography process at a pH of less than 4 advantageously exhibits a shelf-life of at least 36 months, at a temperature in the range from 2 to 8° C.

In a still further aspect the invention relates to a pharmaceutical composition comprising the conjugated protein and pharmaceutically acceptable excipients.

A process for removing residual transglutaminase from peptides or recombinant proteins enzymatically conjugated by microbial transglutaminase (MTGase) to hydrophilic non-immunogenic polymer at a glutamine side-chain through an amidic linkage is hereby described. The resulting purified conjugated peptide or protein is stable against the enzymatic hydrolysis of the amidic bond between the peptide or protein moiety and the hydrophilic polymer and being free from product derived degradation displays the stability required for a drug.

The preferred embodiments of the present invention are illustrated, but not limited in any way, by the following examples concerning a method for purifying Met-G-CSF (filgrastim) enzymatically monopegylated with amidic bond on glutamine135 from contaminating enzyme MTGase by cation exchange chromatography at low pH in order to eliminate any significant deamidation during the shelf-life of the conjugated biodrug.

### EXAMPLES

### Example 1

The MTGase preparation used in the following examples was from batches of commercial enzyme (Activa WM, Ajinomoto) partially purified as described by Scaramuzza et al. (J. Control Rel. 164, 355-363, 2012) to enzymatic activity not lower than 30 unit/mg protein when assayed by the colorimetric hydroxamate procedure with N-α-carbobenzoxy-L-glutaminyl-glycine (N-CBZ-Gln-Gly) and hydroxylamine as substrates according to the method of Folk and Cole (J. Biol. Chem. 241, 5518-5525, 1966).

### Quantitation of Residual MTGase in Met-G-CSF-Gln135-PEG20 kDa

Determination of MTGase contaminating pegylated Met-G-CSF at part per million (ppm) level is performed by evaluating residual MTGase contamination by a modification of a method described by Pasternack R. et al. (Anal. Biochem. 249, 54-60, 1997). Briefly, 1-N-(Benzyloxo carbonyl-L-glutaminyl-glycinyl)-5-N-(5′-N′,N′-dimethylamino-V-naphtalene sulfonly)-diamino, pentane (Z-Gln-Gly-CAD-DNS) is used as MTGase substrate forming a fluorescent conjugated product between glutamine of CBZ-Gln-Gly-CAD-DNS and the amino group of 2-methoxy ethylamine (MED). Five solutions for calibration curve were prepared by mixing 30 μl of a 2.6 mg/ml CBZ-Gln-Gly-CAD-DNS in DMSO/water (9:1) with 20 μl of 1.7 mg/ml MED aqueous solution, 10 μl of DMSO and 20 μl of Tris-HCl— pH 8.0 buffer. To each solution, 100 μl of MTGase standard aqueous solution (respectively 40, 100, 200, 400 and 800 ng/ml) were added.

Sample solutions are prepared as the calibration solutions ones except for the addition of 100 μl (200 ng/ml) of the pegylated Met-G-CSF solution instead of the standard MTGase one.

Sample and reference solutions are incubated at 37° C. for 22 hours and reaction is stopped by adding 100 μl of acetonitrile.

After centrifugation, the surnatants of sample and standard solutions are analyzed by RP-HPLC on a Hypersyl C18, 5 μm, 250×4.6 mm i.d. column equipped with a fluorimetric detector (excitation at 335 nm, emission at 550 nm) eluted at a flow rate of 0.5 ml/min with a 15 minute gradient of acetonitrile 40%→90%; H₂O 60%→10%. The retention time of the conjugated product is about 8.0 minutes, while that of the reagent (CBZ-Gln-Gly-CAD-DNS) is about 7.3 minutes.

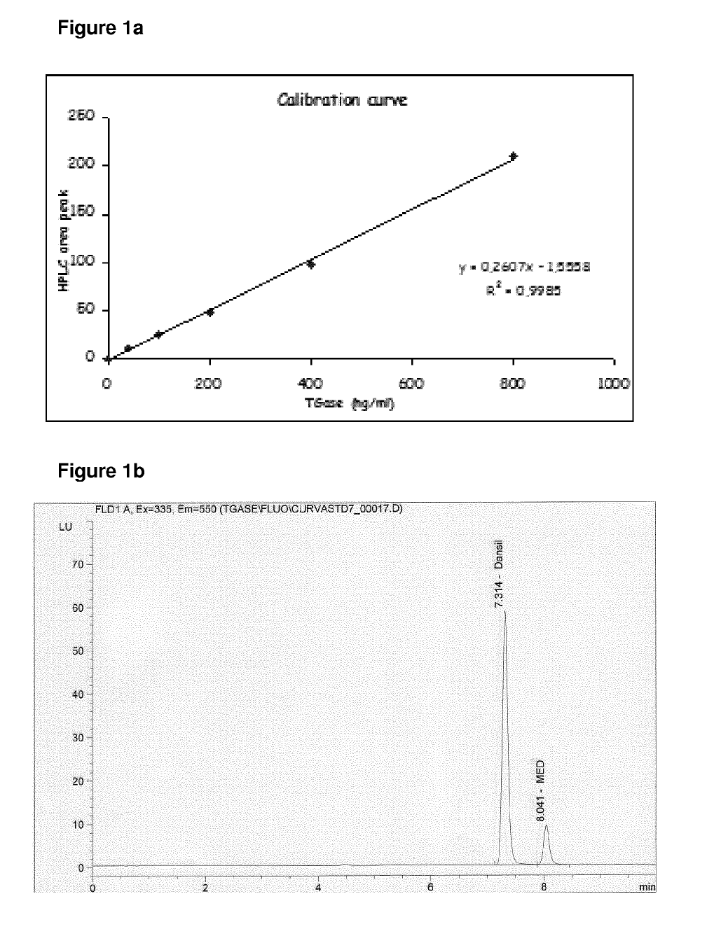

Quantitation is performed by interpolating the area of the CBZ-Gln(Gly-CAD-DNS)-Q-NH-CH2-CH2-O-Me in the sample solution on the calibration curve obtained by incubating CBZ-Gln-Gly-CAD-DNS and 2-methoxyethylamine with different amount of standard MTGase. Table 1 shows the peak areas of duplicate standard solutions, while FIG. 1 reports a typical HPLC separation of the substrate and product of enzymatic reaction of a standard solution together with the obtained calibration curve.

TABLE 1
RP-HPLC fluorimetric assay of MTGase standard solution

| Standard MTGase | MTGase | RP-HPLC area peak* | | |
|---|---|---|---|---|
| ng/ml | U/ml | Sample 1 | Sample 2 | Mean |
| 0 | 0 | 0 | 0 | 0 |
| 40 | 0.0010 | 12.2 | 9.3 | 10.8 |
| 100 | 0.0025 | 26.8 | 24.4 | 25.6 |
| 200 | 0.0050 | 47.6 | 48.9 | 48.3 |
| 400 | 0.0100 | 103.7 | 91.6 | 97.7 |
| 800 | 0.0200 | 215.7 | 204.7 | 209.9 |

*Arbitrary units

### Example 2

### MTGase Catalyzed Met-G-CSF-Gln135-PEG 20 kDa Depegylation: Influence of the Amount of MTGase

1.5 ml of highly purified filgrastim pegylated at Gln135 via MTGase (batch #08105, 10.0 mg/ml, formulated with sorbitol and Tween20 pH 4.5) and also containing 5 ppm of contaminating MTGase are transferred in 6 vials.

To each vial, a suitable amount of MTGase was added in order to obtain a MTGase concentration of 150, 50, 20, 10, 5, 2 ppm.

Each vial was stored in refrigerator (5±3° C.) or in incubator at 25±2° C. Samples were pulled after 1, 2, 3 and 6 months and analyzed by SE-HPLC for evaluating the content of desamidofilgrastim expressed as percentage of total peak areas after dilution 1:40 v/v with acetate buffer 10 mM pH 4.5. Analyses were carried out by injecting 5 μl sample on a Zorbax GF-250, 4 μm, 4.6×250 mm column equipped with an UV detector at 210 nm and maintained at 25° C. Isocratic elution with K2HPO4 63 mM buffer at pH 7 was carried out at a flow rate of 0.250 ml/min.

Results of the analyses are reported in tables 2 and 3, where the actual contents of MTGase was adjusted by adding the residual MTGase detected in the batch #08105.

TABLE 2
Percentage of depegylated product (desamidated filgrastim)
formed during stability study at 5° C. and pH4.5 of filgrastim
pegylated at Gln135 spiked with different amounts of MTGase.

| Time | Amount of MTGase* | | | | | |
|---|---|---|---|---|---|---|
| (months) | 7 ppm | 10 ppm | 15 ppm | 25 ppm | 55 ppm | 155 ppm |
| 0 | <0.1 | <0.1 | <0.1 | <0.1 | <0.1 | <0.1 |
| 1 | 0.1 | 0.5 | 0.0 | 2.2 | 6.4 | 23.1 |
| 2 | 0.3 | 0.9 | 1.6 | 5.6 | 14.9 | 44.7 |
| 3 | 0.4 | 0.8 | 2.6 | 7.7 | 19.8 | 53.3 |
| 6 | 0.4 | 2.2 | 13.2 | — | — | — |

*Sum of originally contained MTGase and MTGase spiking

TABLE 3
Percentage of depegylated product (desamidated filgrastim) formed during
stability study at 25° C. and pH 4.5 of filgrastim pegylated at Gln135
spiked with different amounts of MTGase.

| Time | Amount of MTGase* | | | | |
|---|---|---|---|---|---|
| (months) | 7 ppm | 10 ppm | 15 ppm | 55 ppm | 155 ppm |
| 0 | <0.1 | <0.1 | <0.1 | <0.1 | <0.1 |
| 1 | 0.7 | 2.7 | 4.7 | 33.5 | 76.4 |
| 2 | 1.3 | 5.3 | 8.9 | — | — |

*Sum of originally contained MTGase and MTGase spiking

### Example 3

### MTGase Catalyzed Met-G-CSF-Gln135-PEG 20 kDa Depegylation: Influence of the pH

2 ml of filgrastim pegylated at Gln135 via MTGase (batch #08-BK-01, 15.5 mg/ml) are transferred in 6 vials. The pH of these solutions are adjusted to the target pHs (3.5, 4.0, 4.5, 5.5, 6.5, 7.5) with diluted acetic acid or diluted sodium hydroxide.

100 μl of these solutions are diluted about 1:40 v/v with acetate buffer 10 mM pH4.5 and analysed by SE-HPLC as described in example 2 in order to determine the content of depegylated desamido filgrastim (Time 0 value in Table 4). To the remaining 1.9 ml I, 14.7 μl of MTGase (3 U/ml, 0.1 mg/ml) corresponding to 1.47 μg and 50 ppm were added.

The content of each vial was divided in 10×150 μl aliquots and stored in refrigerator at 5±3° C. Samples were pulled after 1 week, 2 weeks, 1, 2, 3 and 6 months and analyzed by SE-HPLC for evaluating the content of depegylated desamido filgrastim as percentage of total peak areas. Results are shown in Table 4.

TABLE 4
Percentage of depegylated product (desamidated filgrastim)
formed during stability study at 5° C. and at different pH values
of filgrastim pegylated at Gln135 spiked with 50 ppm of MTGase.

| Time | Solution pH | | | | | |
|---|---|---|---|---|---|---|
| (months) | pH 3.5 | pH 4.0 | pH 4.5 | pH 5.5 | pH 6.5 | pH 7.5 |
| 0 | 1.3 | 1.2 | 1.3 | 1.3 | 1.3 | 1.2 |
| 0.25 | 1.2 | 1.3 | 1.9 | 3.2 | 2.6 | 2.0 |
| 0.5 | 1.2 | 1.4 | 2.9 | 6.0 | 4.4 | 2.7 |
| 1 | 1.2 | 1.8 | 4.4 | 10.7 | 7.4 | 3.5 |
| 2 | 1.3 | 2.3 | 7.7 | 19.4 | 12.8 | 5.1 |
| 3 | 1.3 | 2.9 | 11.4 | 29.8 | 18.3 | 6.1 |
| 6 | 1.5 | 4.4 | 19.2 | 49.3 | 27.5 | 8.9 |

As control, solutions of filgrastim pegylated at Gln135 titrated at pH 4 were maintained without MTGase spiking in the same storage condition up to six months and analysed at time intervals as described. Results are reported in table 5.

TABLE 5
Percentage of depegylated product (desamidated filgrastim) formed during
stability at 5° C. and pH 4.0 of filgrastim pegylated at Gln135
without MTGase spiking
Pegylated filgrastin without MTGase spiking

| Time (months) | pH 4 |
|---|---|
| 0 | 1.3 |
| 0.25 | 1.1 |
| 0.50 | 1.2 |
| 1 | 1.3 |
| 2 | 1.4 |
| 3 | 1.2 |
| 6 | 1.2 |

### Example 4

### Preparation of Met-G-CSF-Gln135-PEG 20 kDa by Reaction of Rec-Met-G-CSF and m-PEG-NH2 in the Presence of MTGase

Recombinant Met-G-CSF (Filgrastim) expressed in E. coli, was dissolved in 20 mM potassium dihydrogen phosphate buffer at pH 8.1 at a concentration of about 2 mg/ml. 20 kDa methoxy-polyethylene glycol-amine, catalog no CIAM-20 (Sunbio, Anyang City, South Korea) was then added to the protein solution in a molar ratio of 10:1 mPEG-NH2:Met-G-CSF. After adding MTGase at a final concentration of 0.25 U/ml, the solution was maintained 16 hours at 5±2° C. under gentle agitation. Four pegylation reactions were carried out, with an average pegylation yield of 84.1±5.4% as shown in tables 6, 7, 8 and 9.

TABLE 6
Summary of results of enzymatic pegylation of Met-G-CSF
(reaction n° 1)

| Pegylation reaction N° 1 | Results |
|---|---|
| 0.37 grams Met-G-CSF + | 0.31 grams Met-G-CSF-Q¹³⁵-PEG 20 kDa |
| 46 units MTGase + | Pegylation yield 83.7% |
| 3.7 grams mPEG-NH₂ 20 kDa | Aggregates (by peak area) 1.6% |

TABLE 7
Summary of results of enzymatic pegylation of Met-G-CSF
(reaction n° 2)

| Pegylation reaction N° 2 | Results |
|---|---|
| 0.39 grams Met-G-CSF + | 0.31 grams Met-G-CSF-Q¹³⁵-PEG 20 kDa |
| 49 units MTGase + | Pegylation yield 78.5% |
| 3.9 grams mPEG-NH₂ 20 kDa | Aggregates (by peak area) 4.4% |

TABLE 8
Summary of results of enzymatic pegylation of Met-G-CSF
(reaction n° 3)

| Pegylation reaction N° 3 | Results |
|---|---|
| 1.40 grams Met-G-CSF + | 1.28 grams Met-G-CSF-Q¹³⁵-PEG |
| 175 units MTGase + | 20 kDa |
| 14 grams mPEG-NH2 20 kDa | Pegylation yield 91.5% |
| | Aggregates (by peak area) 1.3% |

TABLE 9
Summary of results of enzymatic pegylation of Met-G-CSF
(reaction n° 4)

| Pegylation reaction N° 4 | Results |
|---|---|
| 0.39 grams Met-G-CSF + | 0.32 grams Met-G-CSF-Q¹³⁵-PEG |
| 49 units MTGase + | 20 kDa |
| 3.9 grams mPEG-NH2 20 kDa | Pegylation yield 82.8% |
| | Aggregates (by peak area) 2.8% |

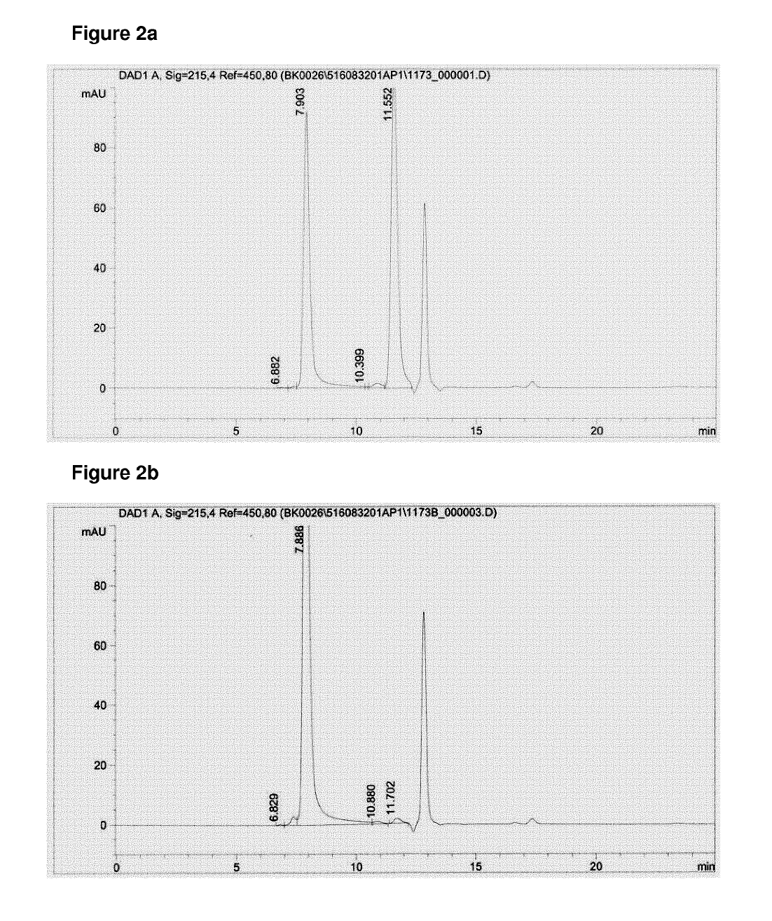

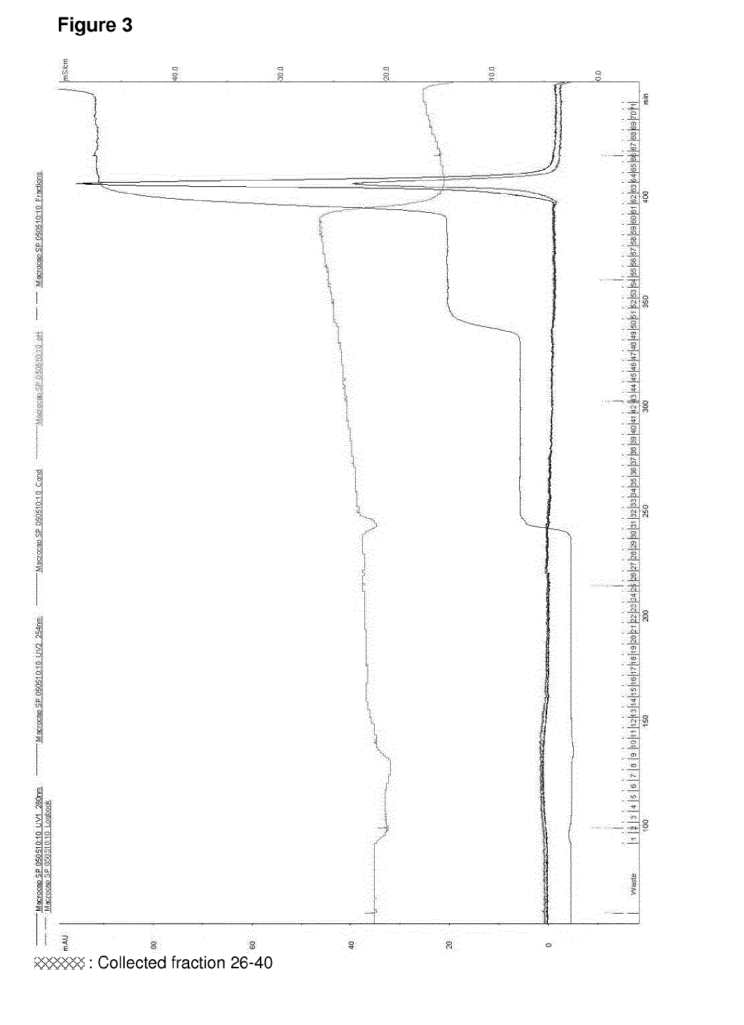

SE-HPLC of reaction mixture after 30 min and 16 hours showed the high formation yield of Met-G-CSF-Gln135-PEG 20 kDa as reported, for example, in FIG. 2.

### Example 5

### Purification of Met-G-CSF-Gln135-PEG 20 kDa at Different pHs

Final solutions of pegylation reactions No 1, 2, 3 and 4 (see example 4) were titrated with 1M acetic acid respectively at pH 5.0, pH 4.5, pH 4.0 and pH 3.5 and separately purified by loading on a cation exchange resin (Macrocap SP) pre-equilibrated with 30 mM sodium acetate buffer titrated at the same pH of loading solution. After column washing with 4 volume column of equilibration buffer, Met-G-CSF-Gln135-PEG 20 kDa was collected by step elution with 200 mM sodium acetate buffered at loading pH. Pooled fractions of purified Met-G-CSF-Gln135-PEG 20 kDa were buffer exchanged on a Sephadex G25 column with 10 mM sodium acetate buffered at the same pH of the elution buffer.

At each purification step, a sample was analysed by SE-HPLC column to determine the reaction yield, the degree of purification and the protein concentration.

An aliquot of the buffer exchanged solution was then concentrated by Amicon ultra membrane (cut-off 10 kDa) in order to obtain a final concentration of about 13 mg/ml. Residual MTGase was determined on concentrated solutions by the RP-HPLC-fluorimetric assay described in the example 1. After each separation, the column was sanitized with 0.5 M sodium hydroxide followed by resin equilibration with 30 mM sodium acetate buffer.

The results of Met-G-CSF-Gln135-PEG 20 kDa purification performed at pH 5, pH 4.5, pH 4.0 and pH 3.5 are summarized in the following tables 10, 11, 12 and 13.

TABLE 10
Summary of Met-G-CSF-Gln135-PEG20 kDa purification at pH 5

| Purification of reaction N° 1 at pH 5.0 | Results |
|---|---|
| Macrocap SP column 1.6 × 28 cm | 0.24 grams grams Met-G-CSF- |
| | Gln135-PEG20 kDa |
| Loading | Purification yield 76.5% |
| 0.31 grams Met-G-CSF-Gln135- | |
| PEG20 kDa | |
| Pool from Macrocap SP column | Aggregates (by area peak) 1.4% |
| 0.24 grams Met-G-CSF-Gln135- | |
| PEG20 kDa | |
| | Residual MTGase 68 ppm |

TABLE 11
Summary of Met-G-CSF-Gln135-PEG20 kDa purification at pH 4.5.

| Purification of reaction N° 2 at pH 4.5. | Results |
|---|---|
| Macrocap SP column 1.6 × 28 cm | 0.29 grams Met-G-CSF-Gln¹³⁵- |
| | PEG20 kDa |
| Loading | Purification yield 93.5% |
| 0.31 grams Met-G-CSF-Gln¹³⁵- | |
| PEG20 kDa | |
| Pool from Macrocap SP column | Aggregates (by area peak) 2.1% |
| 0.29 grams Met-G-CSF-Gln¹³⁵- | |
| PEG20 kDa | |
| | Residual MTGase 36 ppm |

TABLE 12
Summary of Met-G-CSF-Gln135-PEG20 kDa purification at pH 4.0.

| Purification of reaction N° 3 at pH 4.0. | Results |
|---|---|
| Macrocap SP column 5 × 28 cm | 0.90 grams Met-G-CSF-Gln¹³⁵- |
| | PEG20 kDa |
| Loading | Purification yield 70.3% |
| 1.28 grams Met-G-CSF-Gln¹³⁵- | |
| PEG20 kDa | |
| Pool from Macrocap SP column | Aggregates (by area peak) 0.4% |
| 0.90 grams Met-G-CSF-Gln¹³⁵- | |
| PEG20 kDa | |
| | Residual MTGase 5 ppm |

TABLE 13
Summary of Met-G-CSF-Gln135-PEG20 kDa purification at pH 3.5.

| Purification of reaction N° 4 at pH 3.5. | Results |
|---|---|
| Macrocap SP column 1.6 × 28 cm | 0.18 grams Met-G-CSF-Gln¹³⁵- |
| | PEG20 kDa |
| Loading | Purification yield 56.2 |
| 0.32 grams Met-G-CSF-Gln¹³⁵- | |
| PEG20 kDa | |
| Pool from Macrocap SP column | Aggregates (by area peak) |
| 0.18 grams Met-G-CSF-Gln¹³⁵- | 0.4% |
| PEG20 kDa | |
| | Residual MTGase < 3 ppm |

### Example 6

### Preparative Ion-Exchange Separation of MTGase at pH 5.0

6.8 mg of MTGase was diluted to 60 ml in acetate buffer pH5.0 and loaded on a ion-exchange resin Macrocap SP. The column was submitted to step elution with 200 mM sodium acetate buffer at pH 5.0 and the elution profile is reported in FIG. 3.

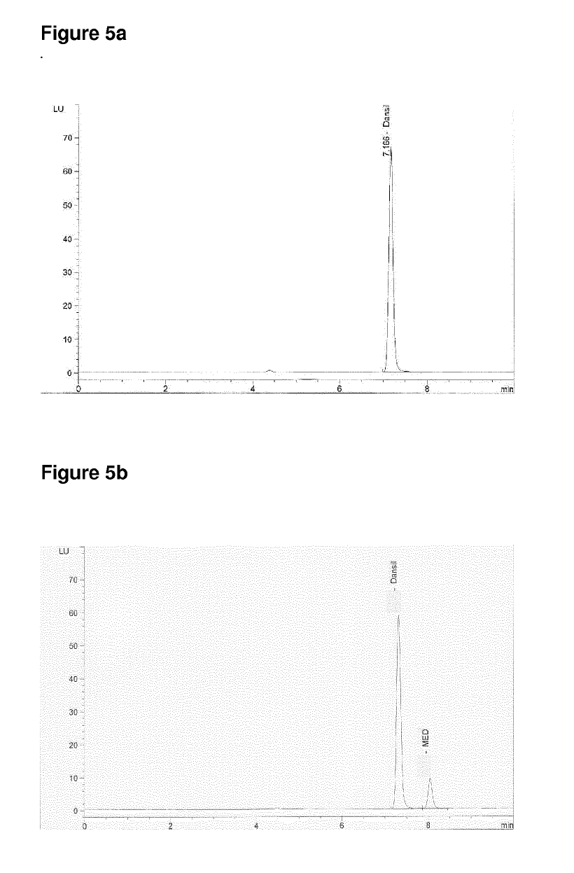

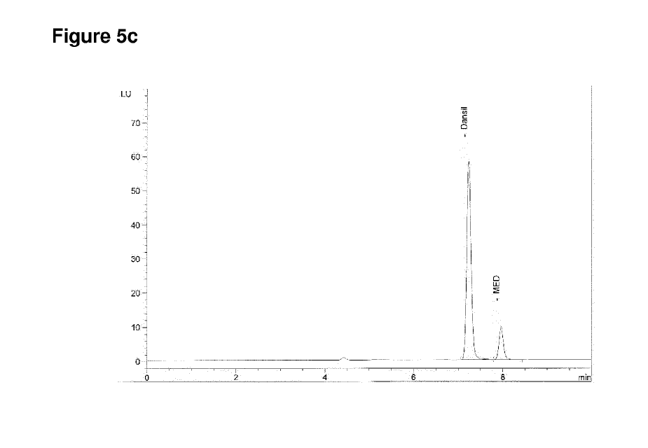

A fraction of 150 ml, corresponding to the elution volume of pegylated Met-G-CSF was collected and 15 ml of this solution were concentrated to 0.6 ml (25 times) by ultrafiltration (Centriprep Ultracell™10; 10.000 MWCO). Quantitation of MTGase was performed using the analytical method described in the example 1 (FIG. 5).

As shown in table 13 no MTGase was detected in this fraction (Limit of Detection=20 ng/ml corresponding to 2.5 ppm in case of pegylated Met-G-CSF co-elution).

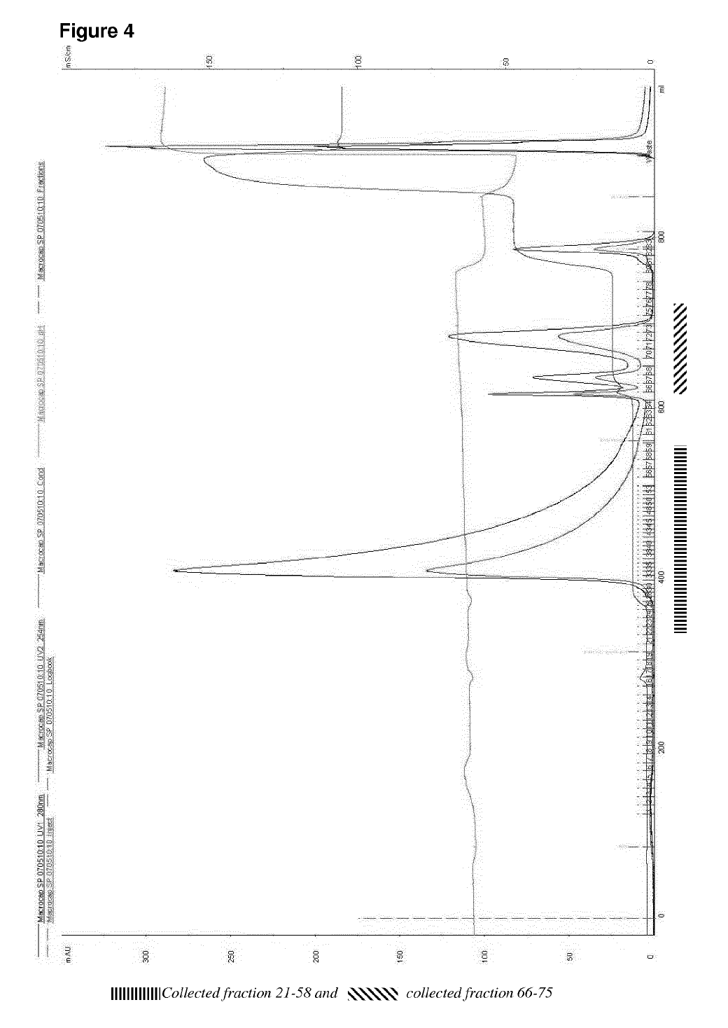

The same amount of MTGase (6.8 mg) was added to a 107.1 mg of purified pegylated Met-G-CSF in 60 ml of acetate buffer pH 5.0. The mixture was loaded on a ion exchange chromatographic column (Macrocap SP). The column was submitted to step elution with 200 mM sodium acetate buffer at pH 5.0 and the elution profile is reported in FIG. 4.

Fractions of about 200 ml corresponding to the elution volume of pegylated Met-G-CSF and of 50 ml, corresponding to the residual unpegylated Met G-CSF were collected and 15 ml of these solutions were concentrated 25 times (to 0.6 ml) by ultrafiltration (Centriprep Ultracell™10; 10,000 MWCO). Quantitation of MTGase was performed using the analytical method described in the example 1 (FIG. 5).

As shown in table 15, the fraction containing the pegylated Met-GCSF was contaminated by 256 ng/ml of MTGase (corresponding to 32 ppm) while the fraction containing the unpegylated Met-G-CSF was contaminated by 1628.4 ng/ml of MTGase and also contained pegylated Met-G-CSF (FIG. 5).

These results demonstrate that MTGase partially bind pegylated Met-GCSF at pH 5.0, rending ineffective the chromatographic purification of pegylated products by ion exchange chromatography.

TABLE 15
Summary of RP-HPLC fluorimetric assay of MTGase in selected
fractions recovered from Macrocap SP separation of MTGase alone and
of MTGase in the presence pegylated Met-GCSF

| | | | | Pegylated- | |
|---|---|---|---|---|---|
| | | RP-HPLC | MTGase | Met-G-CSF | MTGase |
| Experiment | Sample | area peak* | ng/ml | mg/ml | ppm |
| MTGase | Fractions | 0 | <20 | 0 | <2.5* |
| | 26-40 | | | | |
| MTGase + | Fractions | 65.1 | 256 | 8 | 32 |
| pegylated- | 21-58 | | | | |
| Met-G-CSF | Fractions | 69.2 | 1628 | 7.5 | |
| | 66-75 | | | | |

*Arbitrary units

### Example 7

### IE-HPLC Identification of the Depegylated Product

A solution of purified pegylated filgrastim plus 50 ppm of MTGase at pH 4.5 prepared as described in the example 5 was maintained 1 month at 5° C. and analysed by ion exchange HPLC (IE-HPLC) in comparison to a solution of highly purified pegylated Met-G-CSF.

A solution of purified non-pegylated Met-G-CSF was treated with 50 ppm of MTGase, kept at room temperature for 1.5 hours and analysed by IE-HPLC in comparison to a solution of purified Met-G-CSF.

IE-HPLC was carried out on TSK gel DEAE-5PW 10 μm, 7.5 cm length×7.5 mm i.d. column maintained at 25° C. and equipped with UV detection at 215 nm. Elution was carried out with mobile phases A (30 mM Tris-HCl buffer, pH 7.5) and B (30 mM Tris-HCl buffer+0.1M NaCl, pH 7.5) at a flow rate of 0.7 ml/min, according to the following gradient:

| | | | | | |
|---|---|---|---|---|---|
| Time (min): | 0 | 2 | 5 | 15 | 40 |
| % of eluent B: | 0 | 0 | 6 | 13 | 75 |

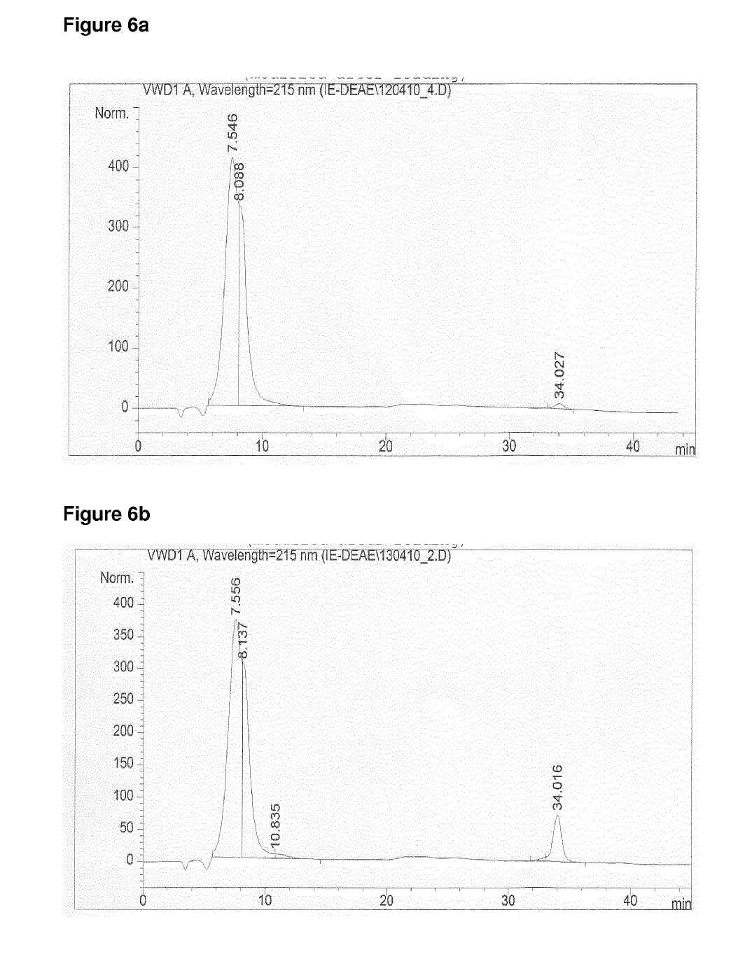

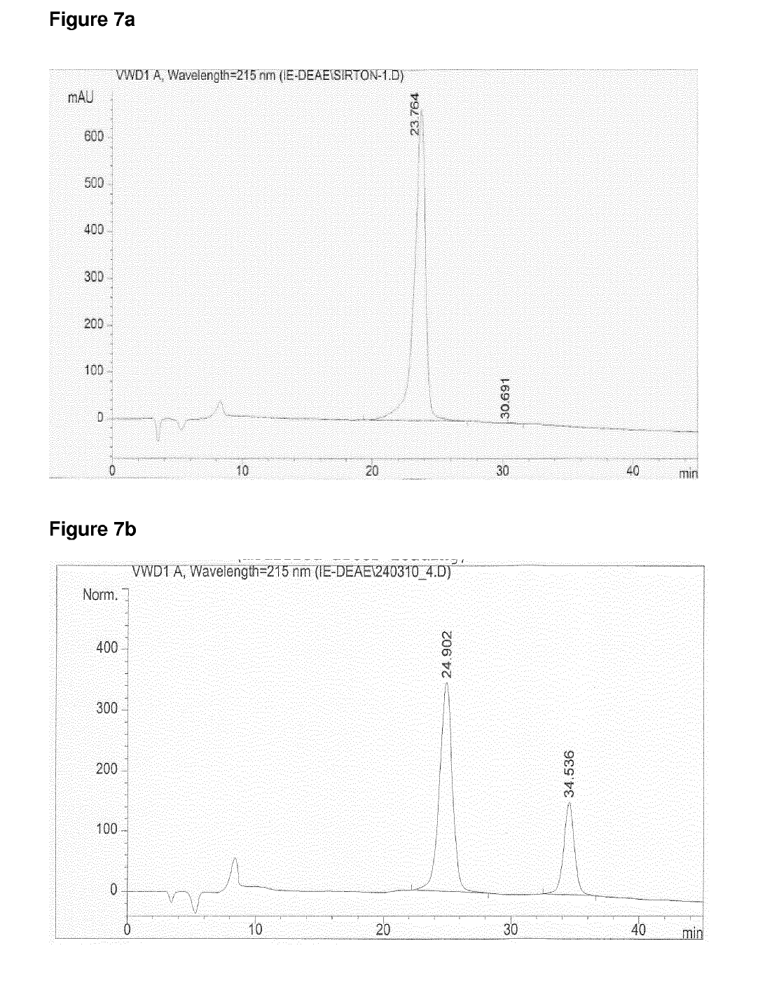

The results, displayed in FIGS. 6 and 7, show that pegylated Met-G-CSF is eluted at the retention time of about 8.2 minutes unretained at the front of the solvent, the depegylated product (desamido Met-G-CSF-Gln¹³⁵Glu) is eluted at about 34 minutes. Non-pegylated Met-G-CSF is eluted with a retention time of about 24 minutes, while an additional peak, similar to that detected in the MTGase treated solution of pegylated Met-G-CSF (retention time of about 34 minutes), is eluted in the chromatogram of the Met-G-CSF treated with M-TGase indicating that the depegylation of r-Met-G-CSF-Q¹³⁵-PEG 20 kDa is accompanied by concomitant deamidation of glutamine 135

From the above description and the above-noted examples, the advantages attained by the process described and obtained according to the present invention are apparent.

1. (canceled)

2. A conjugated protein, obtained by an enzymatic reaction catalyzed by microbial transglutaminase (MTGase), characterized by the fact that the content of residual MTGase in the purified product is not higher than 3 p.p.m., said conjugated protein exhibiting no more than 0.1% of depegylated product after 36 months of storage at a temperature in the range from 2 to 8° C.

3. The conjugated protein according to claim 1, obtained through an enzymatic reaction catalyzed by microbial transglutaminase, between a therapeutic protein and a hydrophilic polymer, preferably a hydrophilic non immunogenic polymer.

4. The conjugated protein according to claim 3, wherein said therapeutic protein is selected from the group consisting of Met-G-CSF, G-CSF, GM-CSF, h-GH, Interferons, interleukins, Fab and scFv antibody fragments, Glucagon, GLP-1, Insulins and derivatives and analogues thereof.

5. The conjugated protein according to claim 3, wherein said hydrophilic non immunogenic polymer is selected from the group consisting of polyethyleneglycol, polyacryloyl morpholine, polyvinyl pyrrolidone, and hydroxyl ethyl starch.

6. The conjugated protein according to claim 3, obtained through the enzymatic reaction catalyzed by microbial transglutaminase, between Met-G-CSF and amino-polyethyleneglycol.

7. Process for the purification of a conjugated protein, obtained by an enzymatic reaction via transglutaminase, by cation exchange chromatography including the steps of,
a. bringing a cation exchange chromatography column to a pH of less than 4;
b. loading the chromatography column with a reaction mixture containing the conjugated protein, having a pH of less than 4 on the column of step a.;
c. eluting the chromatography column of step b. with an eluent having a pH of less than 4, thereby collecting a fraction containing the conjugated protein having a residual microbial transglutaminase content lower or equal to 3 ppm of the total amount of the conjugated protein, said conjugated protein exhibiting no more than 0.1% of depegylated product after 36 months of storage at a temperature in the range from 2 to 8° C.

8. The process according to claim 7, wherein the pH of steps a., b. and c. is in the range of from 3 to 3.9, preferably pH 3.8, and wherein the pH of steps a. and b. is obtained with an acetate buffer, preferably a 30 mM acetate buffer.

9. The process according to claim 7, wherein the pH of step c. is obtained with an acetate buffer, preferably a 200 mM acetate buffer.

10. process according to claim 7, wherein after the loading step b., the chromatography column is washed with an acetate buffer, preferably a 30 mM acetate buffer, in a volume which is of 4 times the volume of the chromatography column.

11. The process according to claim 7, wherein said reaction mixture of step b. is obtained through an enzymatic reaction catalyzed by microbial transglutaminase, between a therapeutic protein and a hydrophilic polymer, preferably a hydrophilic non immunogenic polymer.

12. The process according to claim 11, wherein said therapeutic protein is selected from the group consisting of Met-G-CSF, G-CSF, GM-CSF, h-GH, Interferons, interleukins, Fab and scFv antibody fragments, Glucagon, GLP-1, Insulins and derivatives and analogues thereof.

13. The process according to claim 11, wherein said hydrophilic non immunogenic polymer is selected from the group consisting of polyethyleneglycol, polyacryloyl morpholine, polyvinyl pyrrolidone, and hydroxyl ethyl starch.

14. The process according to claim 7, wherein said conjugated protein is obtained through an enzymatic reaction catalyzed by microbial transglutaminase, between Met-G-CSF and amino-polyethyleneglycol.

15. The process according to claim 7, wherein said conjugated protein exhibits no more than 0.1% of depegylated product after 36 months of storage at a temperature in the range from 2 to 8° C.

16. (canceled)

17. A conjugated protein obtainable by cation exchange chromatography process at a pH of less than 4 according to claim 14, said conjugated protein exhibiting no more than 0.1% of depegylated product after 36 months of storage; at a temperature in the range from 2 to 8° C.

18. A pharmaceutical composition comprising the conjugated protein according to claim 17, and pharmaceutically acceptable excipients.
